(19) United States
(12) Patent Application Publication
(10) Pub. No.: US 2012/0302445 A1
(43) Pub. Date: Nov. 29, 2012

(54) PROCESS FOR RECYCLING POLYMER FINES

(76) Inventors: Henry Rudolph, Krefeld (DE); Thomas Hoefges, Krefeld (DE); Manfred Van Stiphoudt, Kempen (DE); Jörg Harren, Baesweiler (DE); Laurent Wattebled, Dusseldorf (DE); Franck Furno, Heiligenhaus (DE); Frank Soppe, Geldern (DE)

(21) Appl. No.: 13/574,945

(22) PCT Filed: Jan. 18, 2011
(86) PCT No.: PCT/EP11/50570

§ 371 Date: Aug. 10, 2012

(30) Foreign Application Priority Data
Feb. 16, 2010 (DE) 10 2010 008 163.9

(51) Int. Cl. B01J 20/26; A61K 47/00; A01N 25/00; B01J 19/00; B01J 20/30

(52) U.S. Cl. 504/358; 252/194; 422/131; 514/772

(57) ABSTRACT

The present invention relates to a process for producing a water-absorbing polymer structure based on acid group-containing monomers, comprising the process steps of:
i) providing a monomer solution comprising the optionally partly neutralized, acid group-containing monomer;
ii) mixing the monomer solution with fines which consist at least to an extent of 90% by weight, based on the total weight of the fines, of particles having a particle size of less than 850 μm, to obtain a monomer solution mixed with fines;
   wherein the mixing in process step ii) is effected in a mixer in which a first stream of the fines and at least one further stream of the monomer solution are passed from different directions simultaneously to a rotating mixing tool.

The present invention relates to a process for producing a water-absorbing polymer structure based on acid group-containing monomers, to the water-absorbing polymer structure which is based on acid group-containing monomers and is obtainable by this process, to water-absorbing polymer structures based on acid group-containing monomers, to an apparatus for producing water-absorbing polymer structures, to a composite, to a process for producing a composite and to the composite obtainable by this process.

Superabsorbents are water-insoluble crosslinked polymers which are capable of absorbing large amounts of aqueous liquids, especially body fluids, preferably urine or blood, while swelling and forming hydrogels, and of retaining them under pressure. In general, these liquid absorptions are at least 10 times or even at least 100 times the dry weight of the superabsorbents or of the superabsorbent compositions of water. By virtue of these characteristic properties, these polymers find use principally in sanitary articles such as nappies, incontinence products or sanitary towels. A comprehensive overview of superabsorbents and superabsorbent compositions, the use thereof and the production thereof is given by F. L. Buchholz and A. T. Graham (editors) in “Modern Superabsorbent Polymer Technology”, Wiley-VCH, New York, 1998.

The superabsorbents are produced generally by the free-radical polymerization of usually partly neutralized monomers bearing acid groups, in the presence of crosslinkers. Through the selection of the monomer composition, of the crosslinkers and of the polymerization conditions, and of the processing conditions for the hydrogel obtained after the polymerization, it is possible to prepare polymers with different absorption properties. Further options are offered by the preparation of graft polymers, for example using chemically modified starch, cellulose and polyvinyl alcohol according to DE-A-26 12 846.

Frequently, fine powders are added to the monomer solution in order, for example, to influence the properties of the superabsorbent, or in order to recycle particular by-products obtained in the production of superabsorbents. For example, EP-A-0 513 780 describes the mixing of the monomer solution with fine superabsorbent particles which are obtained as a by-product in the production of superabsorbents and can be recycled in this manner. According to the teaching of EP-A-0 513 780, the fine superabsorbent particles are mixed with the monomer solution in simple mixing drums which rotate about a vertical or horizontal axis.

The particular disadvantage of the mixing processes known from the prior art, in which fine powders are mixed with the monomer solution, is, however, that they enable merely inhomogeneous distribution of the fine powders in the monomer solution. This is especially true in the case of fines, for instance fine superabsorbent particles, which can be dispersed in the monomer solution only with difficulty. However, an inhomogeneous distribution of the fine powders in the monomer solution leads to inhomogeneous distribution of the fine powders also in the polymer gel obtained after the polymerization and ultimately also in the superabsorbent obtained after the comminution and drying of the polymer gel. This inhomogeneous distribution of the fine powders in the end product then leads ultimately also to inhomogeneous product properties. In addition to the inhomogeneous distribution of the fines in the monomer solution, the process described in EP-A-0 513 780 is also disadvantageous in that it allows only the incorporation of very small amounts of fine superabsorbent into the monomer solution. Furthermore, the fine superabsorbent particles introduced by means of conventional mixing apparatus settle out very rapidly in the monomer solution. In order to counteract this, it is necessary to use comparatively large amounts of initiator in order to initiate the polymerization rapidly.

It was an object of the present invention to overcome the disadvantages which arise from the prior art in connection with the mixing of fine powders and preferably aqueous monomer solution, especially aqueous acrylic acid solutions.

It was a particular object of the present invention to specify a process for producing water-absorbing polymer structures, with which it is possible to incorporate fines, for example fine superabsorbent particles, into monomer solutions. The process should especially be suitable for recycling fine superabsorbents to obtain superabsorbents with very substantially homogeneous absorption properties. Compared to the processes known from the prior art, it should also enable the incorporation of relatively large amounts of superabsorbent fines, for example of amounts of more than 5% by weight, based on the weight of the monomer solution.

It was another object of the present invention to provide water-absorbing polymer structures which can be used particularly efficiently in hygiene articles with a high superabsorbent content. In this case, the water-absorbing polymers should have not only an advantageously high absorption rate but also particularly high absorption under compressive stress, particularly high retention and particularly high permeability.

A contribution to the solution of the problems stated at the outset is made by a process for producing a water-absorbing polymer structure based on acid group-containing monomers, comprising the process steps of:
i) providing a preferably aqueous monomer solution comprising the optionally partly neutralized, acid group-containing monomer;
ii) preferably continuously mixing the preferably aqueous monomer solution with fines which consist at least to an extent of 90% by weight, more preferably of at least 95% by weight and most preferably of at least 99% by weight, based in each case on the total weight of the fines, of particles having a particle size of less than 850 μm, more preferably less than 600 μm, even more preferably less than 450 μm, even more preferably less than 300 μm and most preferably less than 150 μm, to obtain a monomer solution mixed with fines;
   wherein the mixing in process step ii) is effected in a mixer in which a first stream of the fines and at least one further stream of the monomer solution are passed from different directions simultaneously to a rotating mixing tool.

Completely surprisingly, but no less advantageously for that, it has been found that fines, especially fine superabsorbent particles, can be dispersed particularly homogeneously in monomer solutions using particular mixing apparatus. Compared to the processes known from the prior art for introduction of fines into monomer solutions, it is also possible to use significantly smaller amounts of initiator, since the dispersed fines settle out significantly more slowly. The process according to the invention has been found to be particularly advantageous in cases in which the monomer is polymerized on a belt, i.e. under conditions under which no further mixing of the monomer solution by means of a stirring or kneading apparatus is effected during the polymerization. In the case of dispersion of fine superabsorbent particles in monomer solutions, it has been found, more particularly, that the rate with which water-absorbing polymer structures are capable of absorbing aqueous liquids can be enhanced significantly by adding these fine superabsorbent particles to the monomer solution which is used to produce the water-absorbing polymer structures using this mixing apparatus under well-defined mixing conditions. More particularly, it has been found that the “Free Swell Rate” (FSR) can be enhanced in a controlled manner as a function of the mixing frequency.

Polymer structures preferred in accordance with the invention are fibers, foams or particles, preference being given to fibers and particles and particular preference to particles.

The dimensions of polymer fibers preferred in accordance with the invention are such that they can be incorporated into or as yarns for textiles, and also directly into textiles. It is preferred in accordance with the invention that the polymer fibers have a length in the range from 1 to 500 mm, preferably 2 to 500 mm and more preferably 5 to 100 mm, and a diameter in the range from 1 to 200 denier, preferably 3 to 100 denier and more preferably 5 to 60 denier.

The dimensions of polymer particles preferred in accordance with the invention are such that they have a mean particle size to ERT 420.2-02 in the range from 10 to 3000 μm, preferably 20 to 2000 μm and more preferably 150 to 850 μm. It is especially preferred that the proportion of the polymer particles having a particle size within a range from 300 to 600 μm is at least 30% by weight, more preferably at least 40% by weight and most preferably at least 50% by weight, based on the total weight of the postcrosslinked water-absorbing polymer particles.

In process step i) of the process according to the invention, a preferably aqueous monomer solution comprising the optionally partly neutralized, acid group-containing monomer is first provided, said preferably aqueous monomer solution preferably comprising
a polymerizable, monoethylenically unsaturated monomer (α1) containing an acid group, or a salt thereof, or a polymerizable, monoethylenically unsaturated monomer containing a protonated or quaternized nitrogen, or a mixture of these monomers, particular preference being given to a polymerizable, monoethylenically unsaturated, acid group-containing monomer and greatest preference to acrylic acid,
optionally a monoethylenically unsaturated monomer (α2) polymerizable with the monomer (α1), and
optionally at least one crosslinker (α3).

The monoethylenically unsaturated, acid group-containing monomers (α1) may be partly or fully, preferably partly, neutralized. The monoethylenically unsaturated, acid group-containing monomers have preferably been neutralized to an extent of at least 25 mol %, more preferably to an extent of at least 50 mol % and further preferably to an extent of 50-80 mol %. In this context, reference is made to DE 195 29 348 A1, the disclosure-content of which is hereby incorporated by reference. The neutralization may partly or fully also follow the polymerization. In addition, the neutralization can be effected with alkali metal hydroxides, alkaline earth metal hydroxides, ammonia, and carbonates and bicarbonates. In addition, any further base which forms a water-soluble salt with the acid is conceivable. Mixed neutralization with different bases is also conceivable. Preference is given to neutralization with ammonia and alkali metal hydroxides, particular preference to that with sodium hydroxide and with ammonia.

In addition, in the case of a water-absorbing polymer structure obtainable by the process according to the invention, the free acid groups may predominate, such that this polymer structure has a pH within the acidic range. This acidic water-absorbing polymer structure may be at least partly neutralized by a polymer structure with free basic groups, preferably amine groups, which is basic compared to the acidic polymer structure. These polymer structures are referred to in the literature as “Mixed-Bed Ion-Exchange Absorbent Polymers” (MBIEA polymers) and are disclosed inter alia in WO 99/34843 A1. The disclosure of WO 99/34843 A1 is hereby incorporated by reference and is therefore considered to form part of the disclosure. In general, MBIEA polymers are compositions which comprise firstly basic polymer structures which are capable of exchanging anions, and secondly a polymer structure which is acidic compared to the basic polymer structure and is capable of exchanging cations. The basic polymer structure has basic groups and is typically obtained by the polymerization of monomers (α1) which bear basic groups or groups which can be converted to basic groups. These monomers are in particular those which have primary, secondary or tertiary amines or the corresponding phosphines or at least two of the above functional groups. This group of monomers includes especially ethyleneamine, allylamine, diallylamine, 4-aminobutene, alkyloxycyclines, vinylformamide, 5-aminopentene, carbodiimide, formaldacine, melamine and the like, and the secondary or tertiary amine derivatives thereof.

Preferred monoethylenically unsaturated, acid group-containing monomers (α1) are preferably those compounds which are specified in WO 2004/037903 A2, which is hereby incorporated by reference and is therefore considered to form part of the disclosure, as ethylenically unsaturated, acid group-containing monomers (α1). Particularly preferred monoethylenically unsaturated, acid group-containing monomers (α1) are acrylic acid and methacrylic acid, acrylic acid being the most preferred.

The monoethylenically unsaturated monomers (α2) copolymerizable with the monomers (α1) used may be acrylamides, methacrylamides or vinylamides.

Preferred (meth)acrylamides are, as well as acrylamide and methacrylamide, alkyl-substituted (meth)acrylamides or aminoalkyl-substituted derivatives of (meth)acrylamide, such as N-methylol(meth)acrylamide, N,N-dimethylamino-(meth)acrylamide, dimethyl(meth)acrylamide or diethyl(meth)acrylamide. Possible vinylamides are, for example, N-vinylamide, N-vinylformamide, N-vinylacetamide, N-vinyl-N-methylacetamide, N-vinyl-N-methylformamide, vinylpyrrolidone. Particularly preferred among these monomers is acrylamide.

In addition, it is possible to use water-soluble monomers as monoethylenically unsaturated monomers (α2) copolymerizable with the monomers (α1). In this context, preference is given especially to alkoxy polyalkylene oxide (meth)acrylates such as methoxy polyethylene glycol (meth)acrylates.

Also conceivable as monoethylenically unsaturated monomers (α2) copolymerizable with the monomers (α1) are water-dispersible monomers. Preferred water-dispersible monomers are acrylic esters and methacrylic esters, such as methyl (meth)acrylate, ethyl (meth)acrylate, propyl (meth)acrylate or butyl (meth)acrylate.

The monoethylenically unsaturated monomers (α2) copolymerizable with (α1) may also include methyl polyethylene glycol allyl ether, vinyl acetate, styrene and isobutylene.

The crosslinkers (α3) used are preferably those compounds specified in WO 2004/037903 A2 as crosslinkers (α3). Among these crosslinkers, particular preference is given to water-soluble crosslinkers. The most preferred are N,N′-methylenebisacrylamide, polyethylene glycol di(meth)acrylates, triallylmethylammonium chloride, tetraallylammonium chloride, and allyl nonaethylene glycol acrylate prepared with 9 mol of ethylene oxide per mole of acrylic acid.

In addition to the monomers (α1) and optionally (α2) and optionally the crosslinker (α3), the monomer solution may also include water-soluble polymers (α4). Preferred water-soluble polymers (α4) comprise partly or fully hydrolysed polyvinyl alcohol, polyvinylpyrrolidone, starch or starch derivatives, polyglycols or polyacrylic acid. The molecular weight of these polymers is uncritical provided that they are water-soluble. Preferred water-soluble polymers (α4) are starch or starch derivatives or polyvinyl alcohol. The water-soluble polymers (α4), preferably synthetic water-soluble polymers such as polyvinyl alcohol, can not only serve as the graft base for the monomers to be polymerized. It is also conceivable to mix these water-soluble polymers with the polymer gel only after the polymerization, or with the already dried, water-absorbing polymer gel.

In addition, the monomer solution may also comprise assistants (α5), which assistants include especially the initiators which may be required for the polymerization, or complexing agents, for example EDTA.

Useful solvents for the monomer solution include water, organic solvents or mixtures of water and organic solvents, the selection of the solvent depending especially also on the manner of the polymerization. Very particular preference is given in accordance with the invention to the use of water as the solvent.

The relative amount of monomers (α1) and (α2) and of crosslinkers (α3) and water-soluble polymers (α4) and assistants (α5) in the monomer solution is preferably selected such that the water-absorbing polymer structure obtained by the process is based
to an extent of 20-99.999% by weight, preferably to an extent of 55-98.99% by weight and more preferably to an extent of 70-98.79% by weight on the monomers (α1),
to an extent of 0-80% by weight, preferably to an extent of 0-44.99% by weight and more preferably to an extent of 0.1-44.89% by weight on the monomers (α2),
to an extent of 0-5% by weight, preferably to an extent of 0.001-3% by weight and more preferably to an extent of 0.01-2.5% by weight on the crosslinkers (α3),
to an extent of 0-30% by weight, preferably to an extent of 0-5% by weight and more preferably to an extent of 0.1-5% by weight on the water-soluble polymers (α4),
to an extent of 0-20% by weight, preferably to an extent of 0-10% by weight and more preferably to an extent of 0.1-8% by weight on the assistants (α5), and
to an extent of 0.5-25% by weight, preferably to an extent of 1-10% by weight and more preferably to an extent of 3-7% by weight on water (α6),
   where the sum of the weights (α1) to (α6) is 100% by weight.

Optimal values for the concentration, especially of the monomers, crosslinkers and water-soluble polymers, in the monomer solution can be determined by simple preliminary tests or else inferred from the prior art, especially publications DE 35 03 458 A1, DE 42 44 548 A1, DE 40 20 780 C1, U.S. Pat. No. 4,076,663, U.S. Pat. No. 4,286,082, DE 27 06 135 A1, DE 43 33 056 A1 and DE 44 18 818 A1.

In process step ii) of the process according to the invention, the preferably aqueous monomer solution is then mixed with fines which consist at least to an extent of 90% by weight, more preferably at least to an extent of 95% by weight and most preferably at least to an extent of 99% by weight, based in each case on the total weight of the fines, of particles having a particle size of less than 850 μm, more preferably less than 600 μm, even more preferably less than 450 μm, even more preferably less than 300 μm and most preferably less than 150 μm. The fines more preferably consist at least to an extent of 95% by weight of particles having a particle size of less than 150 μm.

The fines are preferably organic or inorganic fines, it being particularly preferred in accordance with the invention when the fines have a water content of less than 30% by weight, more preferably less than 25% by weight and most preferably less than 5% by weight, based on the total weight of the fines.

The organic fines used may be any particulate organic material which is known to those skilled in the art and is typically used to modify the properties of water-absorbing polymers. The preferred organic fines include cyclodextrins or derivatives thereof, and polysaccharides. Also preferred are cellulose and cellulose derivatives such as CMC, cellulose ethers.

Preferred cyclodextrins or cyclodextrin derivatives are those compounds disclosed in DE-A-198 25 486 at page 3 line 51 to page 4 line 61. The aforementioned section of this published patent application is hereby incorporated by reference and is considered to form part of the disclosure of the present invention. Particularly preferred cyclodextrins are underivatized α-, β-, γ- or δ-cyclodextrins.

The inorganic fines used may be any particulate inorganic material which is known to those skilled in the art and is typically used to modify the properties of water-absorbing polymers. The preferred inorganic fines include carbonates, for instance sodium carbonate, potassium carbonate, ammonium carbonate, magnesium carbonate or calcium carbonate, where the carbonates may optionally be granulated carbonates, and where the carbonates may optionally also be modified, for example encapsulated by means of polyalkylene glycols, as described, for example, in US 2005/0137546 A1, sulfates such as Na₂SO₄, lactates, for instance sodium lactate, silicates, especially framework silicates such as zeolites, or silicates which have been obtained by drying aqueous silica solutions or silica sols, for example the commercially available products such as precipitated silicas and fumed silicas, for example Aerosils having a particle size in the range from 5 to 50 nm, preferably in the range from 8 to 20 nm, such as “Aerosil 200” from Evonik Industries AG, aluminates, titanium dioxides, zinc oxides, clay materials, and further minerals familiar to those skilled in the art, and also carbonaceous inorganic materials.

Preferred silicates are all natural or synthetic silicates which are disclosed as silicates in Holleman and Wiberg, Lehrbuch der Anorganischen Chemie [Inorganic Chemistry], Walter de Gruyter-Verlag, 91^{st}-100^{th} edition, 1985, on pages 750 to 783. The aforementioned section of this textbook is hereby incorporated by reference and is considered to form part of the disclosure of the present invention.

Particularly preferred silicates are the zeolites. The zeolites used may be all synthetic or natural zeolites known to those skilled in the art. Preferred natural zeolites are zeolites from the natrolite group, the harmotome group, the mordenite group, the chabazite group, the faujasite group (sodalite group) or the analcite group. Examples of natural zeolites are analcime, leucite, pollucite, wairakite, bellbergite, bikitaite, boggsite, brewsterite, chabazite, willhendersonite, cowlesite, dachiardite, edingtonite, epistilbite, erionite, faujasite, ferrierite, amicite, garronite, gismondine, gobbinsite, gmelinite, gonnardite, goosecreekite, harmotome, phillipsite, wellsite, clinoptilolite, heulandite, laumontite, levyne, mazzite, merlinoite, montesommaite, mordenite, mesolite, natrolite, scolecite, offretite, paranatrolite, paulingite, perlialite, barrerite, stilbite, stellerite, thomsonite, tschernichite or yugawaralite. Preferred synthetic zeolites are zeolite A, zeolite X, zeolite Y, zeolite P, or the product ABSCENTS.

The zeolites used may be zeolites of the so-called “intermediate” type, in which the SiO₂/AlO₂ ratio is less than 10; the SiO₂/AlO₂ ratio of these zeolites is more preferably within a range from 2 to 10. In addition to these “intermediate” zeolites, it is also possible to use zeolites of the “high” type, which include, for example, the known “molecular sieve” zeolites of the ZSM type, and β-zeolite. These “high” zeolites are preferably characterized by an SiO₂/AlO₂ ratio of at least 35, more preferably by an SiO₂/AlO₂ ratio within a range from 200 to 500.

The aluminates used are preferably the naturally occurring spinels, especially common spinel, zinc spinel, iron spinel or chromium spinel.

Preferred titanium dioxides are pure titanium dioxide in the rutile, anatase and brookite crystal forms, and also iron-containing titanium dioxides, for example ilmenite, calcium-containing titanium dioxides such as titanite or perovskite.

Preferred clay materials are those disclosed as clay materials in Holleman and Wiberg, Lehrbuch der Anorganischen Chemie, Walter de Gruyter-Verlag, 91^{st}-100^{th} edition, 1985, on pages 783 to 785. Particularly the aforementioned section of this textbook is hereby incorporated by reference and is considered to form part of the disclosure of the present invention. Particularly preferred clay materials are kaolinite, illite, halloysite, montmorillonite and talc.

Further inorganic fines preferred in accordance with the invention are the metal salts of the mono-, oligo- and polyphosphoric acids. Among these, preference is given especially to the hydrates, particular preference being given to the mono- to decahydrates and trihydrates. Useful metals include especially alkali metals and alkaline earth metals, preference being given to the alkaline earth metals. Among these Mg and Ca are preferred and Mg is particularly preferred. In the context of phosphates, phosphoric acids and metal compounds thereof, reference is made to Holleman and Wiberg, Lehrbuch der Anorganischen Chemie, Walter de Gruyter-Verlag, 91^{st}-100^{th} edition, 1985, on pages 651 to 669. The aforementioned section of this textbook is hereby incorporated by reference and is considered to form part of the disclosure of the present invention.

Preferred carbonaceous but nonorganic fines are those pure carbons which are mentioned as graphites in Holleman and Wiberg, Lehrbuch der Anorganischen Chemie, Walter de Gruyter-Verlag, 91^{st}-100^{th} edition, 1985, on pages 705 to 708. The aforementioned section of this textbook is hereby incorporated by reference and is considered to form part of the disclosure of the present invention. Particularly preferred graphites are synthetic graphites, for example coke, pyrographite, activated carbon or carbon black.

In a particularly preferred embodiment of the process according to the invention, the fines are, however, water-absorbing fine polymer structures based on acid group-containing monomers (so-called “superabsorbent fines”), which are obtained in transport or screening steps in the course of production and finishing of superabsorbents. In the course of customary possible uses of superabsorbents, the superabsorbents are generally ground to particle sizes of about 150 to about 850 μm. Owing to the brittleness of the material, this operation, however, gives rise to a considerable proportion of particles having a particle size of less than 150 μm. These particles are, however, frequently removed before the use of the superabsorbents, since they have an adverse effect on the permeability properties of the superabsorbent material and additionally promote dust formation when the superabsorbent material, for example, is transported in conveyor systems in the course of production of hygiene articles. In general, this removal of the fines is accomplished by screening.

The water-absorbing fine polymer structures which are based on acid group-containing monomers and are used in process step ii) of the process according to the invention may, with regard to the proportions in the composition of the above-described components (α1) to (α6), be identical to the water-absorbing polymer structures obtainable by the process according to the invention or else different therefrom. In the first case, the water-absorbing fine polymer structures based on acid group-containing monomers originate from a process in which water-absorbing polymer structures have been produced and are identical in terms of chemical composition to those which are being produced in the same process in which the water-absorbing fine polymer structures based on acid group-containing monomers are being added to the monomer solution. In the second case, the water-absorbing fine polymer structures based on acid group-containing monomers originate correspondingly from a process in which water-absorbing polymer structures have been produced and are not identical in terms of chemical composition to those which are being produced in the process in which the water-absorbing fine polymer structures based on acid group-containing monomers are being added to the monomer solution. Furthermore, the fine polymer structures based on acid group-containing monomers may be surface postcrosslinked or surface nonpostcrosslinked. Also conceivable is the use of mixtures of surface postcrosslinked and surface nonpostcrosslinked fine polymer structures based on acid group-containing monomers.

The amount of fines, more preferably of water-absorbing fine polymer structures based on acid group-containing monomers, which are mixed with the preferably aqueous monomer solution in process step ii) of the process according to the invention is preferably within a range from 0.1 to 15% by weight, more preferably within a range from 0.5 to 10% by weight and most preferably within a range from 3 to 8% by weight, based in each case on the weight of the preferably aqueous monomer solution.

The process according to the invention is characterized in that the mixing in process step ii) is effected in a mixer in which a first stream of the fines and at least one further stream of the monomer solution are passed from different directions simultaneously to a rotating mixing tool. Such a manner of mixing is implemented in mixers of the “rotor-stator type”. Such rotor-stator systems comprise, in their mixing chamber, a generally cylindrical, non-rotating stator, in the centre of which the likewise preferably cylindrical rotor rotates. Both the walls of the rotor and those of the stator are typically characterized by recesses, for example recesses in the form of slots, through which the mixture of monomer solution and fines is sucked and in this way is exposed to particularly high shear forces.

In this context, it is especially preferred that the first stream of the fines and the at least one further stream of the monomer solution form an angle δ within a range from 60 to 120°, more preferably within a range from 75 to 105°, further preferably within a range from 85 to 95° and most preferably of about 90°. It is also preferred in accordance with the invention that the stream of the mixture of the monomer solution and the fines which leaves the mixer, and the first stream of the fine polymer structures, form an angle ε within a range from 60 to 120°, more preferably within a range from 75 to 105°, even more preferably within a range from 85 to 95° and most preferably of about 90°.

Preferably, the first stream of the fines and the at least one further stream of the monomer solution are passed, preferably simultaneously, to the rotating mixing tool continuously over a period of at least 10 minutes, even more preferably at least 120 minutes and most preferably at least 240 minutes.

Such a manner of mixing can be realized especially by selecting, as the mixer, a mixer with a vertical supply shaft for the fines and with at least one mixing tool which rotates about a vertical axis, and with at least one lateral feed for the preferably aqueous monomer solution. Such a mixer is described, for example, in DE-A-25 20 788 and in DE-A-26 17 612, the disclosure-content of which with regard to the mixing apparatus described therein is hereby incorporated by reference and forms part of the disclosure of the present invention.

In the case of use of the mixing apparatus described in DE-A-25 20 788, the fines are conducted by means of a fall tube which serves as the supply shaft into a nozzle body which surrounds the lower end of this fall tube and narrows in the vertical direction from the top downwards, towards the mouth of the fall tube, but also maintains a lateral distance from this mouth. The preferably aqueous monomer solution to be mixed in is conducted through the upper, relatively wide region of this nozzle body, such that the monomer solution in the lower region of the fall tube at first flows approximately vertically from the top downwards like the fines, and in doing so increases its flow rate as a result of the reduction in the cross section. This nozzle body extends in the vertical direction a little beyond the lower end of the fall tube, below which there is a mixing plate which rotates about a vertical axis as the mixing tool. This mixing plate preferably rotates within a stator, both the mixing plate and the stator being provided with recesses through which the mixture of fines and monomer solution is sucked.

In the case of use of the mixing apparatus described in DE-A-26 17 612, the fines are conducted past a valve-like and adjustable occlusion body arranged in the vertical feed stream of these fines, below which there is an approximately coaxial arrangement of nozzles which thus again act in the vertical direction from the top downwards, from which the preferably aqueous monomer solution emerges for mixing with the fines. Under a rotating mixing basket provided with mixing tools below, this premixture passes through a funnel to a second mixing chamber in which are arranged further nozzles for supply of the monomer solution, which are directed obliquely from the top downwards. Below this, the overall mixture passes to a further mixing tool in the form of a mixing plate or mixing basket which at first throws the overall mixture radially outwards, before it can then be drawn off again in the downward direction.

In a particularly preferred embodiment of the process according to the invention, the mixing tool is configured in the form of paddles which act from the top downwards in the vertical conveying direction of the fines, or has such paddles, the supply of the preferably aqueous monomer solution to the mixing chamber being arranged obliquely or radially from the side in the conveying region of the mixing tool, and the region above the mixing tool being free from a supply of monomer solution. Such a mixing apparatus, which is particularly preferred in accordance with the invention, is described in DE-A-196 29 945, the disclosure-content of which with regard to the mixing apparatus described therein is hereby likewise incorporated by reference and forms part of the disclosure of the present invention.

In this context, it is further preferred that, above the mixing tools or the mixing chamber, a conveying screw which acts in the supply direction of the fine polymer structures and is arranged between the mixing tools and the supply shaft for the fines separates the essentially dry supply shaft from the wet mixing region, i.e. fills the cross section of the supply shaft or the continuation thereof virtually completely—apart from a small tolerance against a wall. In this way, the fines are introduced into the mixing chamber via forced conveying and mixed therein with the monomer solution. The conveying screw may be arranged coaxially on a continuation of the drive shaft for the mixing tools and may also be driven by the drive motor for the mixing tools. It is also particularly advantageous when a dispersing device arranged coaxially on the same drive shaft is arranged below the mixing chamber. This allows even more intense continued mixing of the mixture of the monomer solution and the fines formed in the mixing chamber.

Such mixing apparatus is available, for example, from IKA® Werke GmbH & Co. KG, Staufen, Germany, preference among these mixing apparatuses being given especially to those obtainable under the MHD 2000/4, MHD 2000/05, MHD 2000/10, MHD 2000/20, MHD 2000/30 and MHD 2000/50 names, the greatest preference among these being given to that designated MHD 2000/20. These mixing apparatuses, with which the fines are mixed continuously with the monomer solution, can be operated continuously with a maximum total throughput within a range from 100 up to 40 000 liters per hour, the maximum throughput of fines typically being within a range from 50 up to about 11 000 liters per hour.

In the context of the above-described mixing apparatuses, especially in the context of the mixing apparatus sold under the MHD 2000/20 name, it is especially preferred that the latter is operated with a mixing frequency (i.e. with a frequency with which the mixing tool rotates) within a range from 10 to 100 hertz, more preferably within a range from 20 to 85 hertz and most preferably within a range from 25 to 75 hertz.

In addition to the above-described rotor-stator systems from IKA® Werke, it is also possible in accordance with the invention to use rotor-stator systems from ystral GmbH, Ballrechten-Dottingen, especially the mixing apparatus sold under the “Conti TDS” name, or else rotor-stator systems from Kinematika AG, Luttau, Switzerland, for example the mixing systems obtainable under the Megatron® name.

Typically, the process according to the invention further comprises, as well as the above-described process steps i) and ii), the process steps of:
iii) free-radically polymerizing the optionally partly neutralized, acid group-containing monomer present in the preferably aqueous monomer solution in the presence of a crosslinker to obtain a polymer gel,
iv) optionally comminuting the polymer gel,
v) drying the optionally comminuted polymer gel to obtain water-absorbing polymer structures,
vi) optionally grinding and screening off the water-absorbing polymer structures,
vii) optionally surface postcrosslinking the optionally ground and screened-off water-absorbing polymer structures.

In process step iii) of the process according to the invention, the preferably aqueous monomer solution which has been mixed with the fines and is obtained in process step ii) is free-radically polymerized to obtain a polymer gel.

The solution polymerization can be effected continuously or batchwise. The prior art discloses a broad spectrum of possible variations with regard to reaction conditions, such as temperatures, type and amount of the initiators and of the reaction solution. Typical processes are described in the following patents: U.S. Pat. No. 4,286,082, DE 27 06 135 A1, U.S. Pat. No. 4,076,663, DE 35 03 458 A1, DE 40 20 780 C1, DE 42 44 548 A1, DE 43 33 056 A1, DE 44 18 818 A1. The disclosures are hereby incorporated by reference and are therefore considered to form part of the disclosure.

The polymerization is triggered by an initiator, as is generally customary. The initiators used to initiate the polymerization may be all initiators which form free radicals under the polymerization conditions and are typically used in the production of superabsorbents. Initiation of the polymerization by the action of electron beams on the polymerizable aqueous mixture is also possible. The polymerization can, however, also be triggered in the absence of initiators of the type mentioned above by the action of high-energy radiation in the presence of photoinitiators. Polymerization initiators may be present dissolved or dispersed in a solution of inventive monomers. Useful initiators include all compounds which decompose to free radicals and are known to the person skilled in the art. These include especially those initiators which are already mentioned in WO 2004/037903 A2 as possible initiators.

Particular preference is given to producing the water-absorbing polymer structures using a redox system consisting of hydrogen peroxide, sodium peroxodisulphate and ascorbic acid.

The initiators may in principle be added before, during or after the mixing of the preferably aqueous monomer solution with the fine polymer structures, particular preference being given to the addition of the initiators after the mixing. When initiator systems composed of more than one initiator are used, for example initiator systems consisting of ascorbic acid, sodium peroxodisulphate and hydrogen peroxide, it is preferred that this system is not completed until after it leaves the mixer, by adding at least one of the components of such initiator systems to the mixture of monomer solution and fines only after it leaves the mixing apparatus.

It is also advantageous to free the monomer solution of oxygen before the polymerization, which can be accomplished, for example, by blowing in inert gases, especially by blowing in nitrogen. It is possible in principle to free the monomer solution of oxygen before it enters the mixing apparatus, in the mixing apparatus or else after it leaves the mixing apparatus. It is also conceivable to free the monomer solution of oxygen both before it enters the mixing apparatus and in the mixing apparatus, and optionally also after it leaves the mixing apparatus.

In addition, in a particular embodiment of the process according to the invention, it is preferred when at least process steps ii) and iii) are effected continuously. This can be accomplished by continuously mixing the monomer solution with the fines by means of the above-described mixing apparatus and then polymerizing the mixture thus obtained continuously, by supplying this mixture to a continuous polymerization apparatus, for example a polymerization belt system.

In process step iv) of the process according to the invention, the polymer gel obtained in process step iii) is optionally comminuted, this comminution being effected especially when the polymerization is performed by means of a solution polymerization. The comminution can be effected by means of comminution apparatus known to those skilled in the art, for instance a meat grinder. These process steps too may, just like process steps ii) and iii), be effected continuously.

In process step v) of the process according to the invention, the polymer gel which has optionally been comminuted beforehand is dried. The polymer gel is preferably dried in suitable driers or ovens. Examples include rotary tube ovens, fluidized bed driers, pan driers, paddle driers or infrared driers. It is additionally preferred in accordance with the invention that the polymer gel is dried in process step v) down to a water content of 0.5 to 25% by weight, preferably of 1 to 10% by weight, the drying temperatures typically being within a range from 100 to 200° C. By virtue of the use of belt driers, it is also possible to perform this process step v) continuously.

In process step vi) of the process according to the invention, the water-absorbing polymer structures obtained in process step v), especially when they have been obtained by solution polymerization, can be ground and screened off to the desired particle size specified at the outset. The dried water-absorbing polymer structures are ground preferably in suitable mechanical comminution apparatus, for example a ball mill, whereas the screening-off can be effected, for example, by using screens with suitable mesh size.

In process step vii) of the process according to the invention, the optionally ground and screened-off water-absorbing polymer structures are surface postcrosslinked. For the surface postcrosslinking, the dried and optionally ground and screened-off water-absorbing polymer structures from process step v) or vi), or else the as yet undried but preferably already comminuted polymer gel from process step iii) or iv), is/are contacted with a preferably organic, chemical surface postcrosslinker. Especially when the postcrosslinker is not liquid under the postcrosslinking conditions, it is preferably contacted with the water-absorbing polymer structures or the polymer gel in the form of a fluid comprising the postcrosslinker and a solvent. The solvents used are preferably water, water-miscible organic solvents, for instance methanol, ethanol, 1-propanol, 2-propanol or 1-butanol or mixtures of at least two of these solvents, water being the most preferred solvent. It is additionally preferred that the postcrosslinker is present in the fluid in an amount within a range from 5 to 75% by weight, more preferably 10 to 50% by weight and most preferably 15 to 40% by weight, based on the total weight of the fluid.

The contacting of the water-absorbing polymer structure or of the optionally comminuted polymer gel with the fluid including the postcrosslinker in the process according to the invention is effected preferably by good mixing of the fluid with the polymer structure or the polymer gel.

Suitable mixing units for applying the fluid are, for example, the Patterson-Kelley mixer, DRAIS turbulent mixers, Lödige mixers, Ruberg mixers, screw mixers, pan mixers and fluidized bed mixers, and also continuous vertical mixers in which the polymer structure is mixed at high frequency by means of rotating blades (Schugi mixer). The water-absorbing polymer structures can also be mixed with the fluid comprising the postcrosslinker in a rotating vessel. Such a process is described, for example, in DE-A-10 2007 024 080.

In the process according to the invention, the polymer structure or the polymer gel is contacted in the course of postcrosslinking preferably with at most 20% by weight, more preferably with at most 15% by weight, further preferably with at most 10% by weight, even further preferably with at most 5% by weight, of solvent, preferably water.

In the case of polymer structures in the form of preferably spherical particles, it is additionally preferred in accordance with the invention that the contacting is effected in such a way that only the outer region but not the inner region of the particulate polymer structures is contacted with the fluid and hence the postcrosslinker.

Postcrosslinkers which are used in the process according to the invention are preferably understood to mean compounds which have at least two functional groups which can react with functional groups of a polymer structure in a condensation reaction (=condensation crosslinkers), in an addition reaction or in a ring-opening reaction. Preferred postcrosslinkers in the process according to the invention are those specified in WO 2004/037903 A2 as crosslinkers of crosslinker classes II.

Among these compounds, particularly preferred postcrosslinkers are condensation crosslinkers, for example diethylene glycol, triethylene glycol, polyethylene glycol, glycerol, polyglycerol, propylene glycol, diethanolamine, triethanolamine, polyoxypropylene, oxyethylene-oxypropylene block copolymers, sorbitan fatty acid esters, polyoxyethylene sorbitan fatty acid esters, trimethylolpropane, pentaerythritol, polyvinyl alcohol, sorbitol, 1,3-dioxolan-2-one (ethylene carbonate), 4-methyl-1,3-dioxolan-2-one (propylene carbonate), 4,5-dimethyl-1,3-dioxolan-2-one, 4,4-dimethyl-1,3-dioxolan-2-one, 4-ethyl-1,3-dioxolan-2-one, 4-hydroxymethyl-1,3-dioxolan-2-one, 1,3-dioxan-2-one, 4-methyl-1,3-dioxan-2-one, 4,6-dimethyl-1,3-dioxan-2-one and 1,3-dioxolan-2-one.

Once the polymer structures or the polymer gels have been contacted with the postcrosslinker or with the fluid including the postcrosslinker, they are heated to a temperature in the range from 50 to 300° C., preferably 75 to 275° C. and more preferably 150 to 250° C., such that, preferably as a result of which, the outer region of the polymer structures is more highly crosslinked compared to the inner region (=postcrosslinking), and, when polymer gels are used, they are simultaneously also dried. The duration of the heat treatment is limited by the risk that the desired profile of properties of the polymer structures is destroyed owing to the action of heat.

In addition, it may be advantageous when, before, during or after the performance of process step vii), further surface modifications are carried out, for instance the coating of the optionally already surface postcrosslinked water-absorbing polymer structures with anti-caking agents, with flow assistants, for instance polyethylene glycols, with odor binders, for instance cyclodextrins, tannins, tea extracts or zeolites, or else with permeability enhancers, for instance inorganic powders or aluminum salts. Such modification measures are sufficiently well known from the prior art.

A contribution to the achievement of the objects stated at the outset is also made by a water-absorbing polymer structure based on acid group-containing monomers, obtainable by the process according to the invention, in which preferably water-absorbing fine polymer structures based on acid group-containing monomers are used as fines, said water-absorbing polymer structure preferably having the following properties:
(β1) an absorption rate determined by the test method described herein of more than 0.2 g/g/s, more preferably of more than 0.22 g/g/s, even more preferably of more than 0.24 g/g/s and most preferably within a range from 0.25 to 0.30 g/g/s;
(β2) a bulk density determined to ERT 460.2-02 of more than 530 g/l, more preferably of more than 560 g/l and most preferably of more than 590 g/l.

A contribution to the achievement of the objects stated at the outset is also made by water-absorbing polymer structures based on acid group-containing monomers and having the following properties:
(β1) an absorption rate determined by the test method described herein of more than 0.20 g/g/s, more preferably of more than 0.22 g/g/s, even more preferably of more than 0.24 g/g/s and most preferably within a range from 0.25 to 0.30 g/g/s;
(β2) a bulk density determined to ERT 460.2-02 of more than 530 g/l, more preferably of more than 560 g/l and most preferably of more than 590 g/l.

In a particularly preferred embodiment of the inventive water-absorbing polymer structures, enclosed in the interior of the polymer structures are fines, more preferably water-absorbing fine polymer structures which are based on acid group-containing monomers and consist at least to an extent of 90% by weight, more preferably to an extent of at least 95% by weight and most preferably to an extent of at least 99% by weight, based in each case on the total weight of the fines or of the water-absorbing fine polymer structures based on acid group-containing monomers, of particles having a particle size of less than 150 μm. Such water-absorbing polymer structures are obtainable by a process in which such fines or water-absorbing fine polymer structures based on acid group-containing monomers are added to a preferably aqueous monomer solution before the performance of the polymerization.

A contribution to the achievement of the objects stated at the outset is also made by an apparatus for producing water-absorbing polymer structures, at least comprising
a mixer in which a first stream of fines, preferably of water-absorbing fine polymer structures based on acid group-containing monomers, and at least one further stream of a monomer solution can be passed from different directions simultaneously to a rotating mixing tool, said mixer having an outlet for the mixture of the monomer solution and the fine polymer structures;
a polymerization apparatus connected to the outlet.

The mixers used are preferably those mixing apparatuses which have already been mentioned at the outset as preferred mixing apparatuses in the context of the process according to the invention, while the polymerization apparatuses used may especially be belt polymerizers or a screw extruder. Belt polymerizers are described especially in EP-A-228 638. The polymerization apparatus may also preferably be followed downstream by continuous comminution apparatus and then preferably continuous drying apparatus.

The wording “a polymerization apparatus connected to the outlet” as used herein is intended to express that the mixture of the monomer solution and the fines or the water-absorbing fine polymer structures based acid group-containing monomers which leaves the mixer can be supplied directly to the polymerization apparatus.

A further contribution to the achievement of the objects described at the outset is made by a composite comprising the inventive water-absorbing polymer structures or the water-absorbing polymer structures obtainable by the process according to the invention and a substrate. It is preferred that the inventive polymer structures and the substrate are bonded to one another in a fixed manner. Preferred substrates are polymer films, for example of polyethylene, polypropylene or polyamide, metals, nonwovens, fluff, tissues, wovens, natural or synthetic fibers, or other foams. It is additionally preferred in accordance with the invention that the composite comprises at least one region which includes the inventive water-absorbing polymer structure in an amount in the range from about 15 to 100% by weight, preferably about 30 to 100% by weight, more preferably from about 50 to 99.99% by weight, further preferably from about 60 to 99.99% by weight and even further preferably from about 65 to 99% by weight, based in each case on the total weight of the region of the composite in question, which region preferably has a size of at least 0.01 cm³, preferably at least 0.1 cm³ and most preferably at least 0.5 cm³.

A particularly preferred embodiment of the inventive composite involves a flat composite as described in WO-A-02/056812 as an “absorbent material”. The disclosure-content of WO-A-02/056812, especially with regard to the exact structure of the composite, the basis weight of its constituents and its thickness, is hereby incorporated by reference and constitutes part of the disclosure of the present invention.

A further contribution to the achievement of the objects cited at the outset is provided by a process for producing a composite, wherein the inventive water-absorbing polymer structures or the water-absorbing polymer structures obtainable by the process according to the invention and a substrate and optionally an additive are contacted with one another. The substrates used are preferably those substrates which have already been mentioned above in connection with the inventive composite.

A contribution to the achievement of the objects cited at the outset is also made by a composite obtainable by the process described above, which composite preferably has the same properties as the above-described inventive composite.

A further contribution to the achievement of the objects cited at the outset is made by chemical products comprising the inventive polymer structures or an inventive composite. Preferred chemical products are especially foams, moldings, fibers, foils, films, cables, sealing materials, liquid-absorbing hygiene articles, especially nappies and sanitary towels, carriers for plant growth or fungal growth regulators or active crop protection ingredients, additives for building materials, packaging materials or soil additives.

The use of the inventive polymer structures or of the inventive composite in chemical products, preferably in the aforementioned chemical products, especially in hygiene articles such as nappies or sanitary towels, and the use of the superabsorbent particles as carriers for plant growth or fungal growth regulators or active crop protection ingredients, also make a contribution to the achievement of the objects cited at the outset.

In the case of use as a carrier for plant growth or fungal growth regulators or active crop protection ingredients, it is preferred that the plant growth or fungal growth regulators or active crop protection ingredients can be released over a period controlled by the carrier.

The invention is now illustrated in detail with reference to test methods and non-limiting figures and examples.

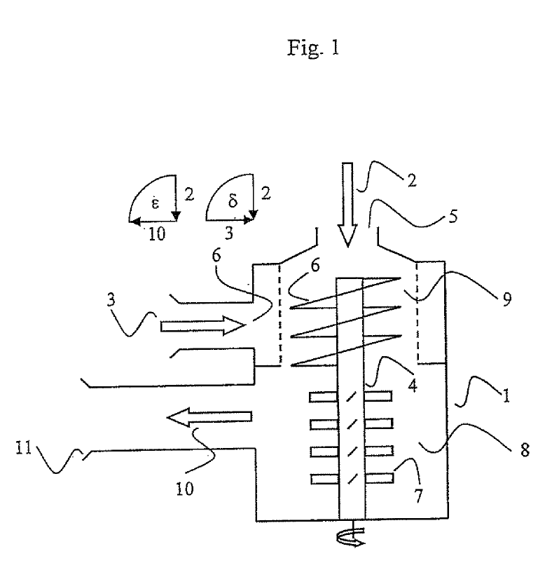

FIG. 1 shows a mixing apparatus which can be used in the process according to the invention for mixing of the monomer solution and the fines.

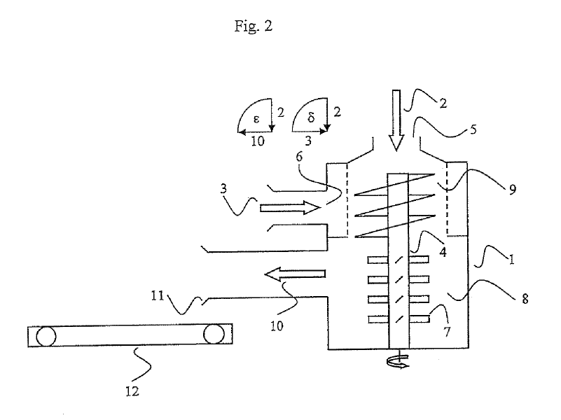

FIG. 2 shows an example of an inventive apparatus for producing water-absorbing polymer structures.

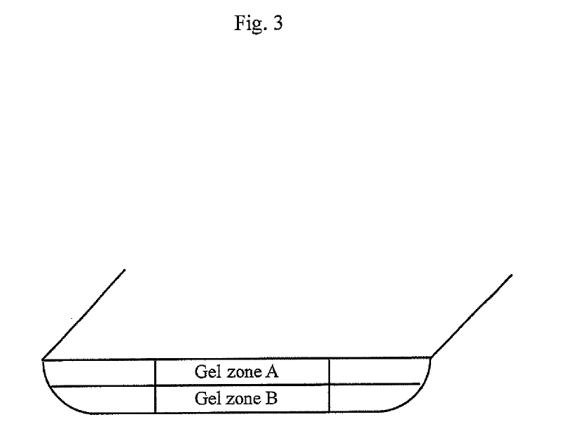

FIG. 3 shows the cross section of the polymer gel obtained in the polymerization in Comparative Example 2 and Example 2 on the polymer belt.

FIG. 1 shows a mixing apparatus 1 usable in the process according to the invention. Via a vertical supply shaft 5, a first stream 2 of fines is passed to a rotating mixing tool 4. Via a lateral feed 6, a further stream 3 of the monomer solution is likewise passed to the rotating mixing tool 4, and streams 2 and 3 enclose an angle δ (in FIG. 1, this angle has a value of about 90°). Within a conveying region 9, the rotating tool 4 is configured, for example, as a conveying screw in order to convey the fines downwards into the mixing chamber 8. In the mixing chamber 8, there is intensive mixing of the fines and of the monomer solution. For this purpose, the mixing tool 4 in the mixing chamber 8 has paddles 7 which act from the top downwards (i.e. paddles which convey the mixture of the fines and the monomer solution downwards as the mixing tool 4 rotates). Below the mixing chamber 8, a dispersion apparatus may also be mounted (not shown in FIG. 1).

Via a lateral outlet 11, the mixture of the fines and the monomer solution is removed from the mixer 1, the stream 10 of this mixture and the stream 2 of the fines forming the angle ε (in FIG. 1, this has a value of about 90°).

In FIG. 2, the above-described mixing apparatus is connected to a belt polymerizer 12 such that the mixture of the fines and the monomer solution leaving via the lateral outlet 11 can be applied directly to a polymerization belt.

### Test Methods

### Free Swell Rate—FSR

The absorption rate was determined via the measurement of the Free Swell Rate (FSR) by the test method described in EP-A-0 443 627 on page 12.

### Centrifuge Retention Capacity—CRC

The CRC was determined according to EDANA test method WSP 241.2-05 (EDANA=European Disposables and Nonwovens Association).

### EXAMPLES

### Comparative Example 1

### Incorporation of SAP Fines in a Monomer Solution by Means of a Conventional Mixing Vessel

12 000 kg of a monomer solution comprising 32% by weight of acrylic acid which had been neutralized to an extent of 75 mol %, 0.3% polyethylene glycol-300 diacrylate, 0.3% allyloxy polyethylene glycol acrylic ester, 3% SAP fines (particles with a particle size of less than 200 μm, which had been colored by means of methylene blue for better identification), and, based on the amount of monomer, 400 ppm of sodium peroxodisulphate, 50 ppm of H₂O₂ and 10 ppm of ascorbic acid, were initially charged.

The mixing vessel was a cylindrical plastic vessel having an internal diameter of 22 cm. Up to the monomer outlet of the vessel, which was at a height of 23.5 cm, there is thus a volume of 8.93 liters. The mixing was effected with a commercial laboratory stirrer at approx. 800 rpm and an anchor stirrer. H₂O₂ and sodium peroxodisulphate were supplied to the mixing vessel; the ascorbic acid was not added until the outlet.

### Example 1

### Incorporation of SAP Fines in a Monomer Solution by Means of an IKA Mixer

6000 kg of a monomer solution comprising 32% by weight of acrylic acid which had been neutralized to an extent of 75 mol %, 0.3% polyethylene glycol-300 diacrylate, 0.3% allyloxy polyethylene glycol acrylic ester, 400 ppm of sodium peroxodisulphate, 50 ppm of H₂O₂ and 10 ppm of ascorbic acid were mixed in an IKA mixer with 3% SAP fines (particles with a particle size of less than 200 μm, which had again been colored by means of methylene blue for better identification), based on the amount of monomer.

The IKA mixer was the MHD 2000/05 model from IKA, which was characterized by a motor speed of 3000/min, a drive speed of 5800/min and a peripheral speed of 22.8 m/sec. This mixer was operated with the 1SA DN 50 feed screw obtainable from IKA, the injector jacket F and the generator PP, which is composed of the rotor PP and stator 2G obtainable from IKA and had a diameter of about 80 mm.

The settling behavior of the fines in the monomer solution was observed in the mixtures obtained in Comparative Example 1 and in Example 1. It was found that the fines introduced by means of the process according to the invention in Example 1 settled out significantly more slowly than those in Comparative Example 1. Therefore, it would be possible to use a smaller amount of initiator to polymerize the monomer solution in Example 1, since the fines settle out more slowly.

### Comparative Example 2

The mixture of monomer solution and SAP fines obtained in Comparative Example 1 was polymerized on a polymerization belt. The initiation temperature in the polymerization was 10° C.

After the polymerization, the gel strands were observed visually in cross section in order to analyze the homogeneity of the distribution of the methylene blue-colored SAP fines. Thereafter, gel samples were taken from two different regions of the cross section (gel zone A: top middle; gel zone B: lower middle, see FIG. 3). These gel samples were ground in a meat grinder with a 5 mm perforated plate, and then dried at 150° C. for 1.5 hours. Finally, the samples were ground and screened off to a particle size within a range from 150 to 850 μm. The CRC and FSR values of the polymers particles thus obtained were determined.

### Example 2

The procedure was as in Comparative Example 2, except that the mixture of monomer solution and SAP fines obtained by means of the IKA mixer in Example 1 was polymerized on the polymerization belt.

As the results in the table which follows show, when the CRC and FSR characteristics are compared, metered addition by means of a mixing vessel reveals significant differences in the particular samples: the CRC declines from 32.3 g/g (upper sample) to 29.7 g/g (lower sample); at the same time, an FSR rise from 0.21 to 0.45 g/g/sec is evident, caused by greater crosslinking of the lower gel region with the higher proportion of SAP fines.

This effect is even more perceptible in the case of a 5% addition of fines: a CRC decline from 31.9 g/g to 27.5 g/g and an FSR rise from 0.13 to 0.46 g/g/sec are found here. By metered addition by means of the IKA mixer, in contrast, a CRC of approx. 32 g/g and an FSR of approx. 0.20 can be achieved for all samples. This shows that superabsorbents produced by means of the process according to the invention with recycled SAP fines can be obtained with significantly more homogeneous absorption properties.

| | CRC | CRC | FSR | FSR |
|---|---|---|---|---|
| | [g/g] | [g/g] | [g/g/sec] | [g/g/sec] |
| | mixing vessel | IKA mixer | mixing vessel | IKA mixer |
| Gel zone A | 32.3 | 32.5 | 0.21 | 0.23 |
| Gel zone B | 29.7 | 32.1 | 0.45 | 0.21 |

### LIST OF REFERENCE NUMERALS

1 mixing apparatus
2 direction in which the fine polymer structures are supplied to the mixing apparatus
3 direction in which the monomer solution is supplied to the mixing apparatus
4 rotating mixing tool
5 supply shaft for fine polymer structures
6 lateral feed for the monomer solution
7 paddle
8 mixing chamber
9 conveying region
10 direction in which the mixture leaves the mixer
11 outlet for the mixture
12 polymerization apparatus

1. A process for producing a water-absorbing polymer structure based on acid group-containing monomers, comprising the process steps of:
i) providing a monomer solution comprising the optionally partly neutralized, acid group-containing monomer; and
ii) mixing the monomer solution with fines which consist at least to an extent of 90% by weight, based on the total weight of the fines, of particles having a particle size of less than 850 μm, to obtain a monomer solution mixed with fines;
wherein the mixing in process step ii) is effected in a mixer in which a first stream of the fines and at least one further stream of the monomer solution are passed from different directions simultaneously to a rotating mixing tool.

2. The process according to claim 1, wherein the first stream of the fines and the at least one further stream of the monomer solution form an angle δ within a range from 60 to 120°.

3. The process according to claim 1, wherein the mixer has a vertical supply shaft for the first stream of the fines, at least one mixing tool which rotates about a vertical axis and a lateral feed for the at least one further stream of the monomer solution.

4. The process according to claim 3, wherein the mixing tool is configured in the form of paddles which act from the top downwards in the direction of the first stream of the fines, or has such paddles, and the supply of the monomer solution to a mixing chamber is arranged obliquely or radially from the side in a conveying region of the mixing tool, and the region above the mixing tool is free from a supply of monomer solution.

5. The process according to claim 1, wherein the mixing tool rotates with a frequency within a range from 25 to 75 Hz.

6. (canceled)

7. The process according to claim 1, wherein a stream of the monomer solution mixed with the fines leaves the mixer, and the first stream of the fines forms an angle ε within a range from 60 to 120° with the stream of the monomer solution mixed with the fines leaving the mixer.

8. The process according to claim 1, wherein the first stream of the fines and the at least one further stream of the monomer solution are passed simultaneously to the rotating mixing tool over a period of at least 10 minutes.

9. The process according to claim 1, wherein the monomer solution comprises at least one crosslinker as well as the acid group-containing monomer.

10. The process according to claim 1, wherein the fines are mixed with the monomer solution in an amount within a range from 0.1 to 10% by weight, based on the weight of the monomer solution.

11. The process according to claim 1, wherein the fines are water-absorbing polymer structures based on acid group-containing monomers.

12. The process according to claim 1, further comprising the process steps of:
iii) free-radically polymerizing the optionally partly neutralized, acid group-containing monomer present in the monomer solution in the presence of a crosslinker to obtain a polymer gel,
iv) optionally comminuting the polymer gel,
v) drying the optionally comminuted polymer gel to obtain water-absorbing polymer structures,
vi) optionally grinding and screening off the water-absorbing polymer structures, and
vii) optionally surface postcrosslinking the optionally ground and screened-off water-absorbing polymer structures.

13. A water-absorbing polymer structure based on acid group-containing monomers, obtainable by the process according to claim 1.

14. The water-absorbing polymer structure based on acid group-containing monomers according to claim 13, wherein the polymer structure has the following properties:
(β1) an absorption rate determined by the test method described herein of more than 0.20 g/g/s; and
(β2) a bulk density determined to ERT 460.2-02 of more than 530 g/l.

15. A water-absorbing polymer structure based on acid group-containing monomers and having the following properties:
(β1) an absorption rate determined by the test method described herein of more than 0.20 g/g/s; and
(β2) a bulk density determined to ERT 460.2-02 of more than 530 g/l.

16. The water-absorbing polymer structure based on acid group-containing monomers according to claim 15, wherein fines which consist at least to an extent of 90% by weight, based on the total weight of the fines, of particles having a particle size of less than 150 μm are enclosed in the interior of the polymer structures.

17. The water-absorbing polymer structure based on acid group-containing monomers according to claim 16, wherein the fines are water-absorbing polymer structures based on acid group-containing monomers.

18. An apparatus for producing water-absorbing polymer structures, comprising:
a mixer in which a first stream of fines and at least one further stream of a monomer solution can be passed from different directions simultaneously to a rotating mixing tool, said mixer having an outlet for the mixture of the monomer solution and the fines; and
a polymerization apparatus connected to the outlet.

19. The apparatus according to claim 18, wherein the polymerization apparatus is a belt polymerizer or a screw extruder.

20. A composite comprising a polymer structure based on acid group-containing monomers according to claim 13 and a substrate.

21. A process for producing a composite, wherein a polymer structure based on acid group-containing monomers according to claim 13 and a substrate and optionally an assistant are contacted with one another.

22. A composite obtainable by a process according to claim 21.

23. Foams, moldings, fibers, foils, films, cables, sealing materials, liquid-absorbing hygiene articles, carriers for plant growth and fungal growth regulators, packaging materials, soil additives or building materials, comprising a polymer structure based on acid group-containing monomers according to claim 13.

24. The use of a polymer structure based on acid group-containing monomers according to claim 13 in foams, moldings, fibers, foils, films, cables, sealing materials, liquid-absorbing hygiene articles, carriers for plant growth and fungal growth regulators, packaging materials, soil additives, for controlled release of active ingredients or in building materials.
